(19) United States
(12) Patent Application Publication
(10) Pub. No.: US 2011/0158862 A1
(43) Pub. Date: Jun. 30, 2011

(54) ESCALATOR HANDRAIL STERILIZER

(75) Inventors: Hyun Kwang Kim, Chungcheongbuk-do (KR); In Ki Yun, Daegu (KR); Young Man Kim, Gyeonggi-do (KR); Dong Sun Min, Gyeonggi-do (KR)

(73) Assignees: Hyun Kwang Kim, Chungcheongbuk-do (KR); In Ki Yun, Daegu (KR)

(21) Appl. No.: 13/060,009

(22) PCT Filed: Aug. 20, 2009
(86) PCT No.: PCT/KR09/04653

§ 371 Date: Mar. 14, 2011

(30) Foreign Application Priority Data
Aug. 21, 2008 (KR) KR1020080081923

(51) Int. Cl. A61L 2/10

(52) U.S. Cl. 422/292

(57) ABSTRACT

The present invention is an escalator handrail sterilizer which is installed close to an inlet or outlet of an escalator handrail, and cleans and sterilizes the handrail moving in or out. The sterilizer comprises a case which is prepared for surrounding the escalator handrail, a chemical spray unit which is prepared in one end inside the case to spray chemicals on the handrail moving in, an ultraviolet ray irradiation unit which is prepared in the other end inside the case to project ultraviolet rays on the chemical-sprayed handrail, a drying unit which dries the chemicals sprayed on the handrail, a control unit which controls the chemical spray unit, the UV irradiation unit and the drying unit, and a cover which is prepared in both ends of the case in order to prevent foreign materials from flowing into the case.

### TECHNICAL FIELD

The present invention relates to an escalator handrail sterilizer and, more particularly, to a handrail sterilizer for maintaining an escalator handrail in a hygienic and clean state.

### BACKGROUND ART

An escalator is a transport device for carrying passengers up or down. Since the escalator has superior transport capacity (6,000-9,000 persons/hr, about 10 times that of an elevator) to an elevator and can carry a number of passengers in a short time, the escalator is widely used in department stores, hotels, subways, airports, and the like.

FIG. 5 is a side section view of a typical escalator.

Referring to FIG. 5, the typical escalator includes steps 1 circulating along a predetermined track to carry a passenger, a handrail 3 disposed at either side of the steps 1 and serving as a handgrip, and a drive unit 5 for driving the steps 1 and the handrail 3.

When the drive unit 5 is operated, the step 1 and the handrail 3 circulate at a predetermined speed along the track so that the escalator carries passengers. Therefore, the passengers on the escalator can be safely moved to a destination while grasping the handrail 3.

In use of the escalator, many passengers usually grip the handrail 3 of the escalator to prevent an accident. Accordingly, there are many problems as follows in terms of hygiene.

First, the surface of the handrail 3 is easily soiled by various foreign matter, such as sweat or the like secreted from the hands of passengers, thereby detracting from the appearance of the handrail 3.

Second, since the handrail 3 of the escalator is frequently grasped by passengers as described above, the handrail 3 suffers from severe bacterial contamination. For reference, although toilets are generally considered to be less sanitary than handrails, research has shown that handrails are contaminated with much more bacteria than toilets.

Various attempts have been made to solve such problems and manual operation of directly wiping the handrail 3 using detergent and a cloth is generally used as the most representative method.

However, since this operation is manually carried out, it has low efficiency and the person in charge of cleaning the handrail may miss certain portions of the handrail. Further, since manually cleaning the handrail only acts to remove foreign matter from the handrail, sterilization of the handrail is not achieved through such manual cleaning. As a result, although the handrail appears clean to the naked eye, bacteria still remain on the handrail even after the operation.

### DISCLOSURE

### Technical Problem

The present invention is directed to solving such problems, and one aspect of the invention is to provide an escalator handrail sterilizer for maintaining an escalator handrail in a hygienic and clean state.

Another aspect of the invention is to provide an escalator handrail sterilizer capable of completely cleaning and sterilizing opposite ends of a handrail which are not easily cleaned.

A further aspect of the invention is to provide an escalator handrail sterilizer which can be installed to an escalator without disturbing passengers using the escalator.

### Technical Solution

One aspect of the present invention provides an escalator handrail sterilizer which is installed close to an inlet or outlet of an escalator handrail, and cleans and sterilizes the handrail moving into or out of the sterilizer. The sterilizer includes a case disposed to surround the escalator handrail, a chemical spray unit disposed at one inner end of the case to spray chemicals to the handrail entering the case, an ultraviolet (UV) irradiation unit disposed at the other inner end of the case to radiate UV light to the handrail having the chemicals sprayed thereon, a drying unit which dries the chemicals on the handrail, a control unit which controls the chemical spray unit, the UV irradiation unit and the drying unit, and a cover disposed at either end of the case to prevent foreign matter from flowing into the case.

### Advantageous Effects

According to embodiments of the present invention, the escalator handrail sterilizer enables complete sterilization using chemicals and UV light, thereby maintaining the escalator handrail in a hygienic and clean state. Further, a case including a chemical spray unit and a UV irradiation unit is disposed to surround the handrail, thereby enabling complete cleaning and sterilization of opposite sides of the handrail which are not easily cleaned manually. Moreover, the escalator handrail sterilizer is disposed close to an inlet or outlet of the escalator handrail, thereby allowing passengers to use the escalator without any discomfort.

### DESCRIPTION OF DRAWINGS

FIG. 1 is a perspective view of an escalator handrail sterilizer in accordance with an exemplary embodiment of the invention;

FIG. 2 is an exploded perspective view of the escalator handrail sterilizer;

FIGS. 3 and 4 are a cross-sectional view taken along line A-A of FIG. 1 and a cross-sectional view taken along line B-B of FIG. 1; and

FIG. 5 is a side section view of a typical escalator.

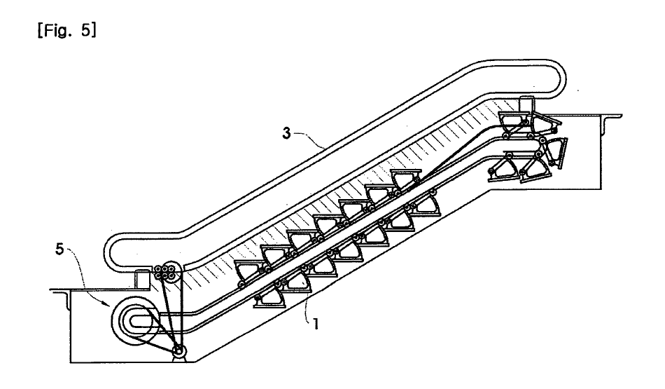

### BEST MODE

Exemplary embodiments of the present invention will now be described with reference to the accompanying drawings.

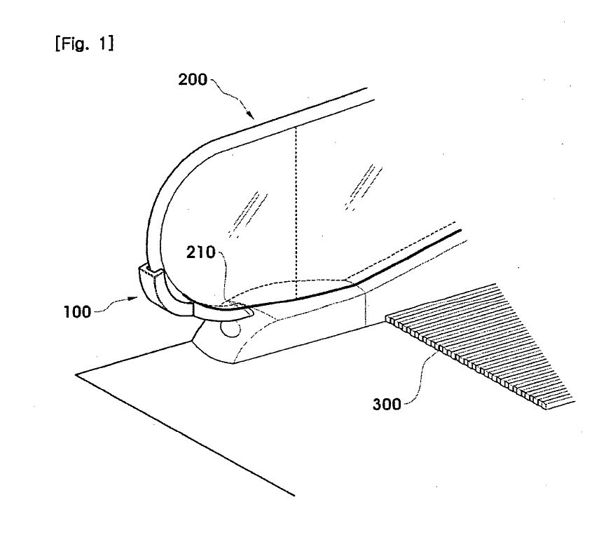

FIG. 1 is a perspective view of an escalator handrail sterilizer in accordance with an exemplary embodiment of the invention.

The escalator handrail sterilizer 100 according to the embodiment permits complete sterilization using chemicals and UV light, thereby enabling maintenance of an escalator handrail 200 (hereinafter, “handrail”) in a hygienic and clean state. The sterilizer 100 may be disposed close to an inlet or an outlet of the handrail 200 according to user need or operator intention so as not to disturb escalator passengers. In this embodiment, the escalator handrail sterilizer 100 is disposed close to an outlet 210 of the handrail 200.

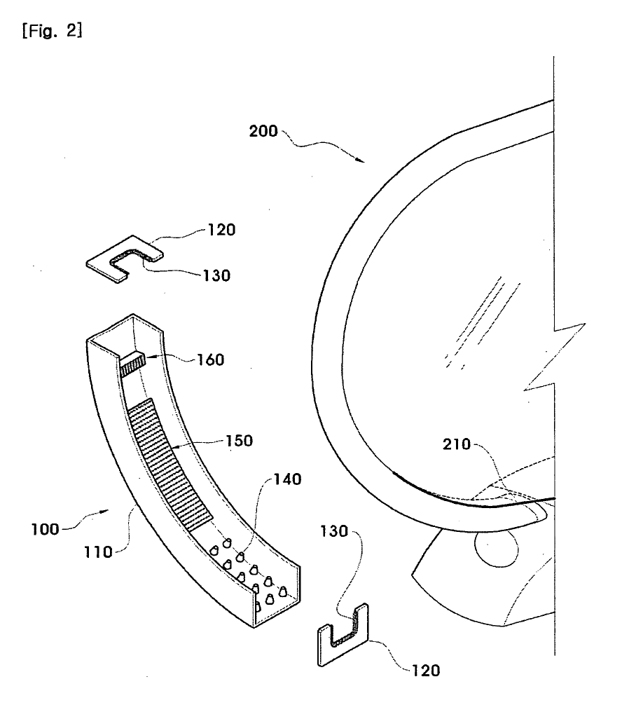

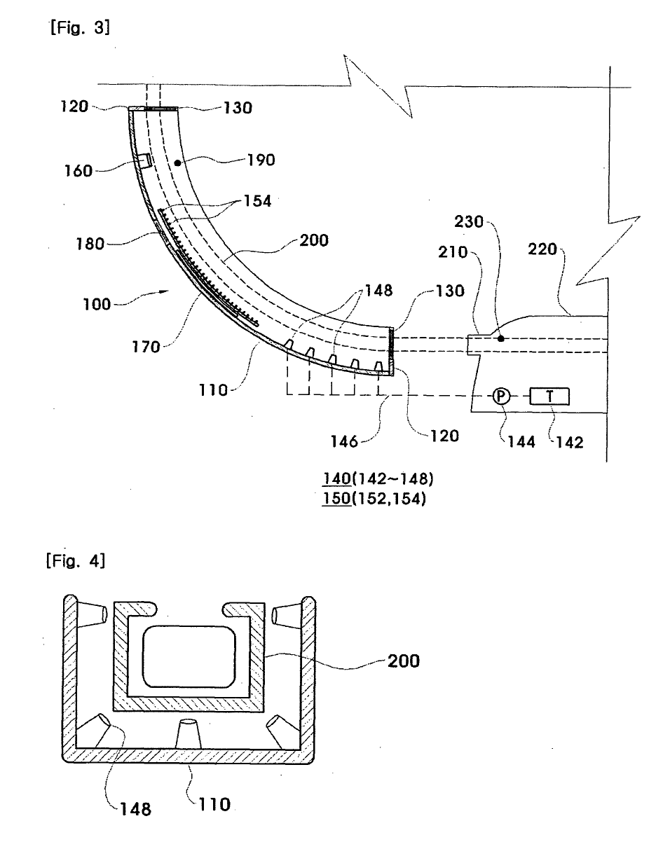

FIG. 2 is an exploded perspective view of the escalator handrail sterilizer, and FIGS. 3 and 4 are a cross-sectional view taken along line A-A of FIG. 1 and a cross-sectional view taken along line B-B of FIG. 1.

As shown in FIG. 2, the escalator handrail sterilizer 100 according to the embodiment includes a case 110 detachably coupled to the outlet of the handrail 200. The case 110 is rounded to surround a portion of the handrail 200 and has opposite ends configured to surround opposite sides of the handrail 200. Therefore, the sterilizer 100 may achieve complete cleaning and sterilization even for the opposite sides of the handrail 200, which do not attract serviceman's attention and are often passed over during cleaning.

The case 110 is provided at a leading end (inlet) and a rear end (outlet) thereof with covers 120 to prevent foreign matter from flowing into the case 110. Further, each of the covers 120 is provided with a brush 130 at a portion contacting the handrail 200 to remove foreign matter from the surface of the handrail 200.

Inside the case 110, a chemical spray unit 140 is disposed at one inner end of the case 110, through which the handrail 200 moves into the case 110, to sterilize the handrail 200 using chemicals and a UV irradiation unit 150 is disposed at a middle section of the case 110 to sterilize the handrail 200 using UV light. Further, the case 110 is provided with a drying unit 160 at the other end thereof, through which the handrail 200 moves out of the case 110, to dry the chemicals on the surface of the handrail 200. Further, the case 110 is provided at an inner wall of the middle section thereof with a control unit 170 (see FIG. 3) to control the chemical spray unit 140, the UV irradiation unit 150 and the drying unit 160.

Next, the respective components 140 to 170 inside the case 100 will be described in more detail with reference to FIGS. 2 to 4.

The chemical spray unit 140 includes a tank 142 for storing chemicals, a pump 144 for pumping the chemicals, a pipe 146 for carrying the chemicals, and nozzles 148 for spraying the chemicals onto the handrail 200. Since the tank 142 and the pump 144 have large volumes, the tank 142 and the pump 144 are disposed inside an escalator housing 220 instead of inside the case 110. Further, a plurality of nozzles 148 is provided to allow the chemicals to reach deep portions at the opposite sides of the handrail 200 and arranged at constant intervals along the inner wall of the cover 110. Advantageously, five nozzles 148 may be arranged at constant intervals along the inner wall of the cover 110, as shown in FIG. 4.

Here, the chemicals used in the chemical spray unit 140 may be various kinds of germicides or disinfectants. For example, the chemicals may be any one selected from alcohol for disinfection, chlorhexidine, para-chloro meta xylenol (PCMX), glutaraldehyde, surfactants, and the like. In this case, the germicides or disinfectants are desirably used at concentrations satisfying regulations and laws concerning use thereof.

The UV irradiation unit 150 includes a printed circuit board 152 (PCB) disposed on the inner wall of the cover 110 and a plurality of light emitting diodes (LEDs) 154 mounted on the PCB 152. The PCB 152 may be a flexible printed circuit board (FPCB) which has elasticity to be applied to the case 110 having a rounded shape, and the LEDs 154 may be arranged at a narrow width to irradiate overlapped areas. Further, the UV irradiation unit 150 including the PCB 152 and the LEDs 154 may be contained in a waterproof material to prevent influence of the chemicals sprayed from the chemical spray unit 140.

At this time, ultraviolet light emitted from the LEDs 154 is UVC capable of sterilizing the handrail 200 and has a wavelength in the range of 100-280 nm UV light is present outside violet of the visible spectrum and has a wavelength of 10-400 nm, which is shorter than that of the visible spectrum. Such UV light is generally classified into UVA (UV-A), UVB (UV-B), and UVC (UV-C) according to the wavelength range. Specifically, UV light in the wavelength range of 315-400 nm is referred to as UVA (UV-A), UV light in the wavelength range of 280-315 nm is referred to as UVB (UV-B), and UV light in the wavelength range of 100-280 nm is referred to as UVC (UV-C). UVA (UV-A) is used for general exploration or forgery detection, UVB (UV-B) is used in medicine or skin care, and UVC (UV-C) is used for sterilization or disinfection equipment.

The drying unit 160 is disposed inside the outlet of the case 110 to dry the chemicals deposited on the surface of the handrail 200. When the handrail 200 is rotated at low speed, the chemicals can be removed from the handrail 200 by drying only with heat from the UV irradiation unit 150. However, when the handrail 200 is rotated at high speed or continuously used for a long period of time, it is insufficient to dry the chemicals from the surface of the handrail only with the heat from the UV irradiation unit 150. Accordingly, the drying unit 160 is positioned inside the outlet of the case 110 immediately before the handrail 200 is exposed to the outside, and provides hot air to the handrail 200 in order to dry the handrail 200. The drying unit 160 has the same configuration as that of a general drying unit, and a detailed description thereof will thus be omitted herein.

The control unit 170 is disposed on the inner wall of the middle section of the case 110, as described above, and is electrically connected to the chemical spray unit 140, the UV irradiation unit 150 and the drying unit 160 to control operation of these components 140 to 160. Here, the operation of the chemical spray unit 140, the UV irradiation unit 150 and the drying unit 160 may be controlled according to operation and speed of the handrail 200. In this case, it is necessary to provide a detection sensor 230 in order to detect the operation and speed of the handrail 200. However, since a typical escalator includes a detection sensor 230 to measure speeds of the handrail 200 and steps 300 (see FIG. 1), this detection sensor 230 may be used to detect the operation and moving speed of the handrail 200. Obviously, when the escalator does not include such a detection sensor, a separate detection sensor may be provided to the sterilizer.

The case 110 is provided with a heat dissipation unit 180 to dissipate heat from the UV irradiation unit 150 and the drying unit 160 to the outside. The heat dissipation unit 180 allows the interior of the case 110 to be maintained at constant temperature, thereby preventing failure of the respective components 140 to 170 and proliferation of bacteria. For this purpose, the case 110 is further provided with a temperature sensor 190 to measure the internal temperature of the case 110.

The heat dissipation unit 180 is also controlled by the control unit 170, which determines operation of the heat dissipation unit 180 by comparing a preset temperature with the temperature of the case 110 measured by the temperature sensor 190.

Cleaning and sterilization of the handrail 200 using the escalator handrail sterilizer 100 according to the embodiment will be described hereinafter.

When an escalator is operated to circulate the handrail 200 and the steps 300, the detection sensor 230 senses operation of the escalator and informs the control unit 170 of the operation. When receiving a signal indicating the operation of the escalator from the detection sensor 230, the control unit 170 operates the chemical spray unit 140, the UV irradiation unit 150 and the drying unit 160 to clean and sterilize the handrail 200.

First, the brush 130 disposed at the leading end (inlet) of the case 110 removes foreign matter from the surface of the handrail 200. The handrail 200 having the foreign matter removed therefrom enters the case 110, faces the chemical spray unit 140, and is primarily sterilized by alcohol for disinfection, chlorhexidine, para-chloro meta xylenol (PCMX), glutaraldehyde, surfactants, or the like, which is sprayed from the chemical spray unit 140. At this time, the control unit 170 regulates the spraying amount of chemicals according to the speed of the handrail 200.

After primary sterilization by the chemical spray unit 140, the handrail 200 is secondarily sterilized by the UV irradiation unit 150. Here, the LEDs 154 of the UV irradiation unit 150 are arranged at a narrow width to irradiate overlapping areas, thereby further improving sterilization of the handrail by UV light.

After the primary and secondary sterilization of the handrail through the chemical spray unit 140 and the UV irradiation unit 150, the chemicals on the surface of the handrail 200 are dried using the drying unit 160. As described above, when the handrail 200 is rotated at low speed, the chemicals can be removed from the handrail 200 by drying only with heat from the UV irradiation unit 150. However, when the handrail 200 is rotated at high speed or continuously used for a long period of time, it is necessary to dry the handrail 200 using the drying unit 160.

Then, the handrail 200, having been subjected to cleaning, sterilization and drying through the aforementioned processes, is exposed outside the case 110 and can be grasped by a passenger. Therefore, the escalator handrail sterilizer 100 enables the handrail 200 to be maintained in a hygienic and clean state.

As the escalator handrail sterilizer 100 is operated for a long period, the internal temperature of the case 110 increases due to heat from the UV irradiation unit 150 and the drying unit 160. In this case, the control unit 170 operates the heat dissipation unit 180 to dissipate heat to the outside based on the internal temperature of the case 110 measured by the temperature sensor 190 inside the case 110. Accordingly, the case 110 may be maintained at a constant temperature.

Although some embodiments have been provided to illustrate the invention in conjunction with the drawings, it will be apparent to those skilled in the art that these embodiments are given by way of illustration only, and that various modifications, changes, alterations and equivalent embodiments can be made without departing from the spirit and scope of the invention. Therefore, the scope of the invention should be limited only by the accompanying claims.

1. An escalator handrail sterilizer comprising:
a case disposed to surround the escalator handrail;
a chemical spray unit disposed at one inner end of the case to spray chemicals to the handrail entering the case;
a UV irradiation unit disposed at the other inner end of the case to radiate UV light to the handrail having the chemicals sprayed thereon; and
a cover disposed at either end of the case to prevent foreign matter from flowing into the case.

2. The sterilizer of claim 1, further comprising: a drying unit which dries the chemicals on the handrail.

3. The sterilizer of claim 2, further comprising: a control unit which controls the chemical spray unit, the UV irradiation unit, and the drying unit.

4. The sterilizer of claim 3, wherein the cover is provided with a brush to remove foreign matter from a surface of the handrail.

5. The sterilizer of claim 4, wherein the sterilizer is disposed close to an inlet or outlet of the handrail.

6. The sterilizer of claim 1, wherein the case is provided with a heat dissipation unit.

7. The sterilizer of claim 1, wherein the chemical spray unit comprises a tank for storing chemicals, a pump for pumping the chemicals, a pipe for carrying the chemicals, and a plurality of nozzles for spraying the chemicals to the handrail, the nozzles being arranged at constant intervals along the inner wall of the cover.

8. The sterilizer of claim 1, wherein the UV irradiation unit comprises a PCB disposed on an inner wall of the cover and a plurality of LEDs mounted on the PCB.

9. The sterilizer of claim 8, wherein the UV irradiation unit is contained in a waterproof material.

10. The sterilizer of claim 8, wherein ultraviolet light emitted from the LEDs has a wavelength in the range of 100-280 nm.

11. The sterilizer of claim 10, wherein the PCB comprises a flexible printed circuit board (FPCB).

12. The sterilizer of claim 1, wherein the controller controls a spraying amount of chemicals and a UV wavelength according to a speed at which the handrail moves.
